(19) United States
(12) Patent Application Publication
(10) Pub. No.: US 2009/0044308 A1
(43) Pub. Date: Feb. 19, 2009

(54) SAUNA GARMENT FOR STIMULATING SWEAT EXCRETION

(76) Inventor: HSIN-MAO HSIEH, Pingtung City (TW)

Correspondence Address: Rabin & Berdo, P.C., Suite 500, 1101 14th Street, Washington, DC, 20005, US

(21) Appl. No.: 11/838,949

(22) Filed: Aug. 15, 2007

(51) Int. Cl. A41D 1/00; A61F 7/00; H05B 1/00

(52) U.S. Cl. 2/69; 219/211; 607/108

(57) ABSTRACT

A sauna garment for stimulating sweat excretion includes a body, a heating device, a tube and a sweat collecting device. The body is mounted with far infrared micro particles and has an inlet. The heating device is mounted on the body and has a heating member. The heating device is connected into the body with the tube. When someone activates the sauna garment, the heating device heats and blows air into the sauna garment. The micro particles in the body are also heated and radiate far infrared rays. The far infrared rays stimulate a human body to secrete sweat. The sauna garment is made of flexible material so is highly portable.

### BACKGROUND OF THE INVENTION

1. Field of the Invention

The present invention relates to a sauna garment for stimulating sweat excretion, especially to sauna garment embedded with far infrared particles. The far infrared particles can be stimulated to radiate far infrared rays by heating air in the sauna garment. The far infrared rays stimulate sweat excretion and improve metabolism of a human body.

2. Description of the Related Art

Many health benefits are associated with sauna, including sweat excretion improving skin condition and raised temperature stimulating circulation and metabolism of a human body.

However, saunas require special equipment that is large and bulky. Therefore, home sauna equipment has been designed on a reduced scale. The home sauna equipment is smaller than regular sauna equipment but is still not portable. Therefore, the conventional home sauna equipment must be installed and remain in one location, which is very inconvenient for highly mobile people since the home sauna equipment is awkward to move. Therefore, a small and highly portable sauna device is necessary in the field.

### SUMMARY OF THE INVENTION

One objective of the present invention is to provide a sauna garment that is small and portable.

To achieve the objective, Sauna garment in accordance with the present invention includes a body, a heating device, a tube and a sweat collecting device. The body is mounted with far infrared micro particles and has an inlet. The heating device is mounted on the body and has a heating member. The heating device is connected to the body with the tube. When someone activates the sauna garment, the heating device heats and blows air into the sauna garment. The micro particles in the body are also heated and radiate far infrared rays. The far infrared rays raise the temperature of a human body and stimulate sweat excretion. The sauna garment is made of flexible material so is highly portable.

Other objectives, advantages and novel features of the invention will become more apparent from the following detailed description when taken in conjunction with the accompanying drawings.

### BRIEF DESCRIPTION OF THE DRAWINGS

FIG. 1 is a partially exploded perspective front view of sauna garment in accordance with the present invention, with a heating device shown in phantom lines;

FIG. 2 is a partially exploded rear perspective view of the sauna garment in FIG. 1; and

FIG. 3 is an enlarged cross sectional view in partial of heating device in FIG. 1.

### DETAILED DESCRIPTION OF THE PREFERRED EMBODIMENTS

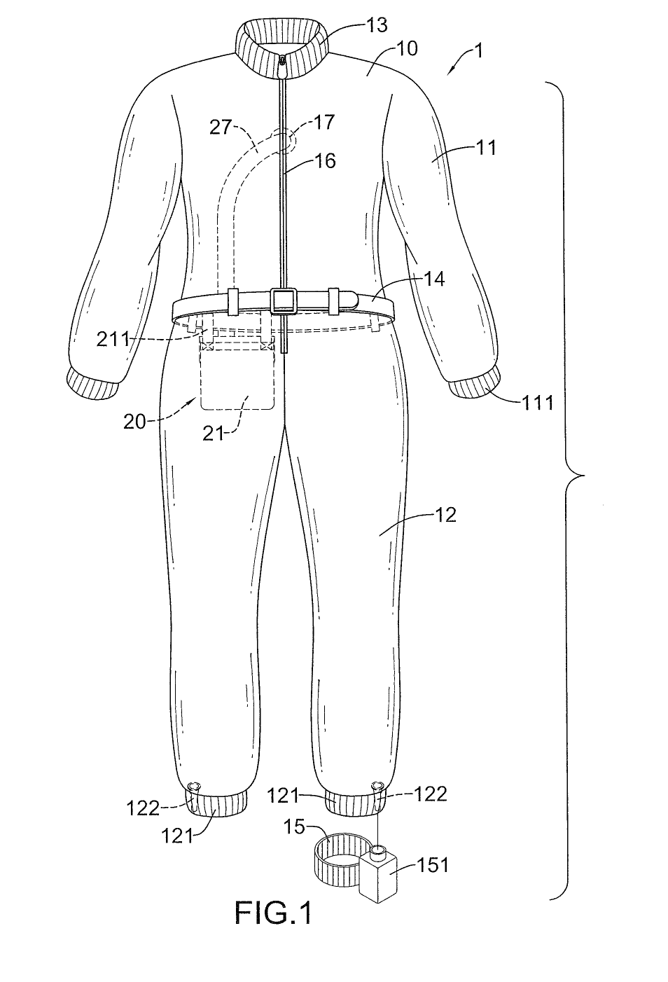

With reference to FIG. 1 a sauna garment in accordance with the present invention comprises a body (10), a heating device (20), a tube (27) and at least one sweat-collecting device.

The body (10) has far infrared micro particles, a front, a neck opening, two sides, a back, a top, a bottom, a bottom opening and an inlet (17). The far infrared micro particles are embedded in the body (10) and may be ceramic or bamboo charcoal powders that absorb heat and radiate far infrared. The neck opening is formed in the top of the body (10) to allow a person's head to extend out of the body. The body (10) may further comprise two sleeves (11), pants (12), at least one resilient hem (111), a closing device (16) and a belt (14).

The sleeves (11) are attached respectively to the sides of the body near the top and each has an opening and a cuff end. Each opening of the sleeves (11) is formed at the cuff end. The pants (12) are attached to the bottom of the body and have two legs, each having an opening and a bottom end. Each opening of the legs of the pants (12) is formed at the bottom end. The pants (12), sleeves (11) and body (10) may be formed together as a jump-suit.

The resilient hems (111) are formed respectively around the openings of the body (10), sleeves (11) and legs of the pants (12) to reinforce the openings.

The closing device (16) is mounted on the body (10) and may be a zip to open and close the sauna garment.

The inlet (17) is formed through the body (10) and may be at the back of the body (10).

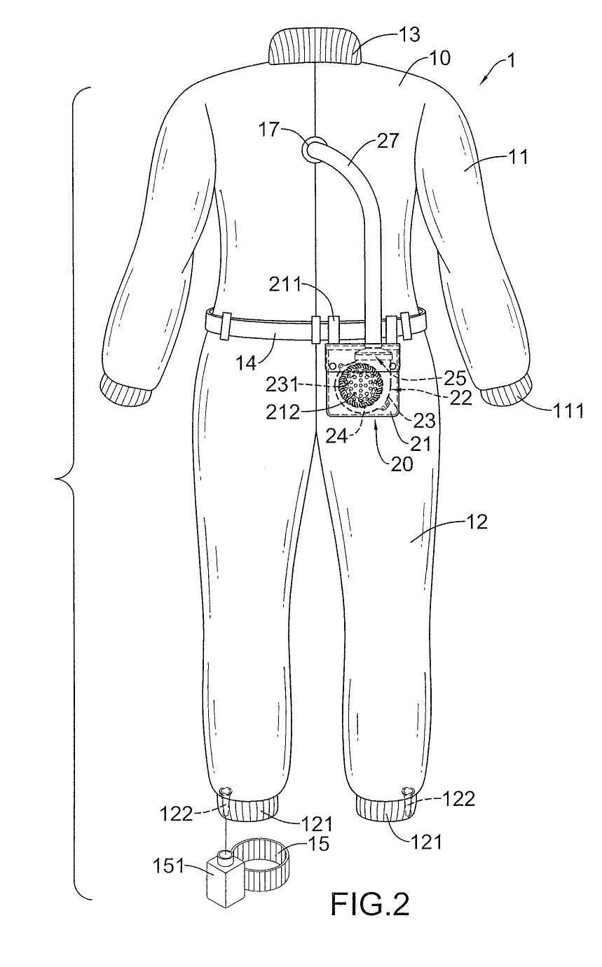

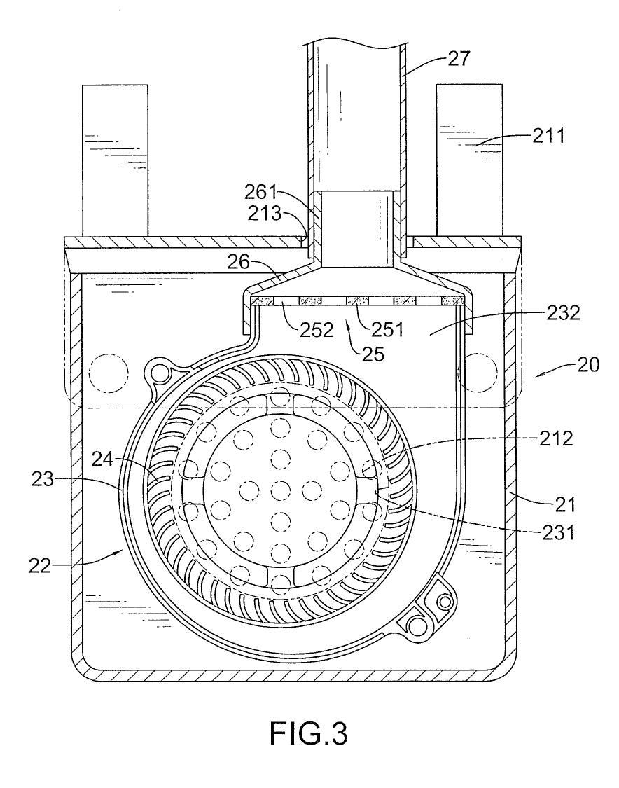

With further reference to FIGS. 2 and 3, the heating device (20) is mounted on the body (10), connected to the inlet (17) and comprises a holder (21), a blower (22), a heating member (25) and may have at least one belt clip (211).

The holder (21) is attached to the body (10) and has an outer surface, an outlet (213) and an inlet (212). The outlet (213) and the inlet (212) are mounted through the outer surface of the holder (21).

The belt clip (211) is attached to the holder (21) and used to clip to the belt (14).The blower (22) is mounted in the holder (21). The blower (22) may be a forced draft fan and may have a case (23), multiple blades (24) rotatably inside of the case (23) and optional connector (26).

The case (23) of the blower (22) may be connected to the connector (26) and has an axle inlet (231) and a side outlet (232). The axle inlet is communicated with the inlet of the holder (21). The side outlet is communicated with the outlet of the holder (21). The connector (26) has two openings. One opening covers the side outlet and the other opening passes through the outlet of the holder (21).

The heating member (25) is mounted on the side outlet of the case (23) and has multiple heating elements (251) and multiple through holes (252).

The heating member (25) uses resistors as heating elements (251) to produce heat. When the blower (22) is operating, air flows through the blower (22) from the inlet of the holder (21) and the axle inlet of the case (23). Then the air flows through the heating member (25) to be heated by the heating elements (251) and flows into the connector (26) to pass the side outlet of the case (23) and the outlet of the holder (21).

The tube (27) is connected between the inlet (17) of the body (10) and the connector (26) of the heating device (20) and communicates the inlet (17) of the body (10) and the outlet of the holder (21).

The sweat collecting device is attached removably to the body (10), may be at the openings of each leg (12) and comprises a conduit (122), a sweat collecting bottle (151) and may have a band (15). The conduit (122) is mounted near the opening, may be in the resilient hem ( 111) at the bottom end of the pants (12). The sweat collecting bottle (151) is connected to the conduit (122) and may be mounted on the band and removably attached to the legs using the band (15).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A sauna garment for stimulating sweat excretion, comprising:
a body being embedded with far infrared micro particles and comprises
a front;
a neck opening formed in the top of the body;
two sides;
a back;
a top;
a bottom;
a bottom opening; and
an inlet formed through the body;
a heating device mounted on the body and connected to the inlet of the body and having
a holder being attached to the body and having an outlet and an inlet;
a blower being mounted in the holder, communicating with the outlet of the holder and the inlet of the holder, and having
an axle inlet communicating with the inlet of the holder; and
a side outlet communicating with the outlet of the holder; and
a heating member being mounted to the side outlet of the blower and having
multiple heating elements; and
multiple through holes;
a tube being connected between the inlet of the body and the side outlet of blower through the outlet of the holder; and
at least one sweat collecting device being attached removably to sauna garment and comprises
a conduit; and
a sweat collecting bottle being connected to the conduit.

2. The sauna garment as claim in claim 1, wherein the blower is a forced draught blower having
a case where the side outlet and the axle inlet are formed, and mounted inside of the holder; and
multiple blades rotatably mounted inside of the case; and
a connector covering the side outlet of the case and projecting through the outlet of the holder to connect the tube.

3. The sauna garment as claim in claim 1, further comprising: a belt attached to the body; and at least one belt clip connected to the holder and attached removably to the belt.

4. The sauna garment as claim in claim 1, wherein the body further comprises:
two sleeves being attached respectively to the sides of the body near the top and each having
a cuff end; and
an opening formed at the cuff end;
pants being attached to the bottom of the body and having
two legs, each having
a bottom end; and
an opening formed at the bottom end;
wherein the body, pants and sleeves are further formed together like a jump-suit.

5. The sauna garment as claim in claim 1, wherein the body further comprises at least one resilient hem being formed around the neck opening of the body to reinforce the opening.

6. The sauna garment as claim in claim 1, the body further comprises multiple resilient hems being formed respectively around the openings of the sleeves, legs of the pants and body to reinforce the openings.

7. The sauna garment as claim in claim 1, further comprising a closing device mounted on the body to open and close the Sauna garment.

8. The Sauna garment as claim in claim 7, wherein the closing device is a zipper.

9. The sauna garment as claim in claim 6, wherein the sweat collecting device further comprises a band being mounted on the sweat collecting bottle.

10. The sauna garment as claim in claim 9, wherein the conduit of the collecting device mounted in the resilient hem at the bottom end of the legs of the pants.
